(19) United States
(12) Patent Application Publication
(10) Pub. No.: US 2012/0053678 A1
(43) Pub. Date: Mar. 1, 2012

(54) INERT HEAT TREATMENT TO IMPROVE COATING ADHESION AND PRODUCT PERFORMANCE CHARACTERISTICS FOR BIODEGRADEABLE COATINGS

(75) Inventors: Umang Anand, Maple Grove, MN (US); Liliana Atanasoska, Edina, MN (US); Pankaj Gupta, Minnetonka, MN (US); Steve Kangas, Woodbury, MN (US)

(73) Assignee: BOSTON SCIENTIFIC SCIMED, INC., Maple Grove, MN (US)

(21) Appl. No.: 13/208,142

(22) Filed: Aug. 11, 2011

Related U.S. Application Data
(60) Provisional application No. 61/377,120, filed on Aug. 26, 2010.

(51) Int. Cl. A61F 2/06; B32B 15/08; B05D 3/02; B32B 15/04; B05D 5/00; B05D 7/00

(52) U.S. Cl. 623/1.46; 427/2.24; 428/457; 428/461

(57) ABSTRACT

An implantable metallic medical device having a dual-layered coating with a primer layer having functional groups capable of forming an organo-metallic complex with iron species and a second biocompatible polymer layer. The primer layer is baked onto the metallic medical device functioning as a tie layer between the surface of the medical device and a second biocompatible polymer layer disposed on the primer layer. Baking improves tie layer adhesion and in some cases provides an organo-metallic complex with the metal substrate. The second biocompatible polymer may can a therapeutic agent.

### CROSS REFERENCE TO RELATED APPLICATIONS

This application claims the benefit of U.S. Provisional Application No. 61/377120, filed on Aug. 26, 2010, the contents of which is hereby incorporated by reference.

### FIELD OF INVENTION

This invention relates to coatings on medical devices such as metallic stents.

### BACKGROUND OF THE INVENTION

Percutaneous transluminal coronary angioplasty (PTCA) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to radially compress against the atherosclerotic plaque of the lesion to remodel the lumen wall. The balloon is then deflated to a smaller profile to allow the catheter to be withdrawn from the patient's vasculature.

Stents are used not only as a mechanical support but also as a vehicle for providing biological therapy. As a mechanical support, stents act as scaffoldings, functioning to physically hold open and, if desired, to expand the wall of the passageway. Typically, stents are capable of being compressed, so that they can be inserted through small vessels via catheters, and then expanded to a larger diameter once they are at the desired location.

Biological therapy can be achieved by applying a biological agent to a stent. Stents coated with a biological agent provide for the local administration of a therapeutic substance at the diseased site.

The present approaches to drug-coated stents incorporates the therapeutic agent into a polymeric solution and then coats the stent, such as described in “Bioactive Agent Release Coating” by Chudzik et al., U.S. Pat. No. 6,214,901. The ideal coating must be able to adhere to the metal stent framework both before and after expansion of the stent, and be able to controllably release the drug at sufficient therapeutic levels for several days, weeks or longer.

All U.S. patents and applications and all other published documents mentioned anywhere in this application are incorporated herein by reference in their entirety.

Without limiting the scope of the invention, a brief summary of some of the claimed embodiments of the invention is set forth below. Additional details of the summarized embodiments of the invention and/or additional embodiments of the invention may be found in the Detailed Description of the Invention below.

A brief abstract of the technical disclosure in the specification is also provided for the purposes of complying with 37 C.F.R. §1.72. The abstract is not intended to be used for interpreting the scope of the claims.

### BRIEF SUMMARY OF THE INVENTION

In some aspects the invention pertains to an implantable device comprising a metallic substrate having a coating over at least a portion of the substrate, the coating comprising:

(a) a drug-free baked primer layer comprising a thermoplastic polymer adjacent the substrate, and

(b) a second layer comprising a biocompatible polymer disposed over at least a portion of the area of said implantable device covered by said primer layer.

Other aspects of the invention pertain to a method of forming a coating on an implantable device comprising:

(a) providing a primer layer comprising a thermoplastic polymer on a metal surface of at least a portion of said implantable device;

(b) baking said implantable device after application of said primer layer at a temperature and time effective to adhere the thermoplastic polymer to the substrate; and

(c) subsequently providing a second layer comprising a biocompatible polymer disposed over at least a portion of the area of the implantable device previously covered by said primer layer.

Still other aspects of the invention pertain to an implantable metal stent comprising a metallic substrate having a coating over at least a portion of the substrate, the coating comprising baked primer layer comprising a polymer capable of forming an organo-metallic complex adjacent the substrate, the polymer having functional groups disposed on a surface of at least a portion of said implantable device which form an organo-metallic complex with iron species of the substrate.

These and other aspects of the invention are described in the drawings, detailed description and claims which follow.

### BRIEF DESCRIPTION OF THE SEVERAL VIEWS OF THE DRAWING(S)

FIG. 1 is a schematic view in detail of a coating on a stent surface.

FIG. 2 shows a flow chart describing a method for applying a primer layer and subsequent coating to the surface of medical device.

FIG. 3A is a baking profile of the primer layer showing the temperature range required to achieve a viscous flow state of the primer layer.

FIG. 3B shows experimental data on the adherence properties of stents with a baked primer layer versus stents without a baked primer layer.

FIG. 3C shows experimental data regarding the performance of stents with a baked primer layer versus stents without a baked primer layer when subjected to flow field tests.

FIG. 3D shows experimental data regarding the performance of stents with a baked primer layer versus stents without a baked primer layer when subjected to wet and dry over expansion tests.

### DETAILED DESCRIPTION OF THE INVENTION

While this invention may be embodied in many different forms, there are described in detail herein specific preferred embodiments of the invention. This description is an exemplification of the principles of the invention and is not intended to limit the invention to the particular embodiments illustrated.

For the purposes of this disclosure, like reference numerals in the figures shall refer to like features unless otherwise indicated. The term “organo-metallic complex” refers to the ionic coordination of a metal ion of the substrate with a functional group of the polymer. The term “acid terminated ends” refers to a substrate used in forming an organo-metallic complex having a free carboxylic acid moiety capable of coordinating a metal ion. The term “ester terminated ends” refers to a substance used in forming an organo-metallic complex having a free ester chemical group capable of coordinating a metal ion. The terms “bake” or “baked” refers to bringing a substance to a temperature where a “viscous flow” state is reached allowing the substance to better adhere to a substrate to which it has previously been applied. The term “viscous flow” is defined as the temperature where the polymer has made the transition from a rubbery crystalline state to a state allowing wetting of the substrate.

The adhesion of polymeric substances to the surface of metallic medical devices that are subjected to deformation stress can be poor. Moreover, the presence of an active ingredient in a polymeric matrix further interferes with the ability of the matrix to adhere effectively to the surface of the device. An increase in the quantity of the active ingredient reduces the effectiveness of the adhesion. High drug loadings of, for example, 10-40% by weight in the coating significantly hinder the retention of the coating on the surface of the device. A primer layer may be employed which serves as a functionally useful intermediary layer between the surface of the device and an active ingredient-containing or reservoir coating. The primer layer provides for an adhesive tie between the reservoir coating and the device which, in effect, would also allow for the quantity of the active ingredient in the reservoir coating to be increased without compromising the ability of the reservoir coating to be effectively contained on the device during delivery and, if applicable, expansion of the device.

It has been found that baking of a thermoplastic layer to the surface of the medical device can significantly improve the coating retention. Without being bound by theory, it is thought that the baking temperature creates a state of viscous flow in the primer layer polymer that creates an environment of better wetting of the stent, thus allowing the polymer to better cover and interact with the surface of the medical device. The baked primer layer may also provide a densification of the primer layer to improve barrier properties of the primer layer to prevent the migration of the drug in the overlying drug-containing layer to the surface of a medical device.

Further, the applicant has found that forming an organo-metallic complex between metal ions in the substrate and functional groups in the polymer play a role in the adhesion improvement of baked primers. Any polymer having a functional group capable of forming an organo-metallic complex can potentially be used. However, polymers having acid or ester end groups are preferred, with a polymer having acid end groups, such as PLGA, giving better adhesion improvements.

In some embodiments PLGA as the primer layer can take advantage of PLGA's acid terminated ends to form an organo-metallic complex with iron species in a metallic medical device. In this way, the primer layer adheres to the metallic surface without the need for an adhesion promoter which can affect the efficacy of a therapeutic agent in the second biocompatible polymer layer. In some embodiments of the invention, the substances for the primer layer have pendant groups or branched polymers with acid or ester end groups.

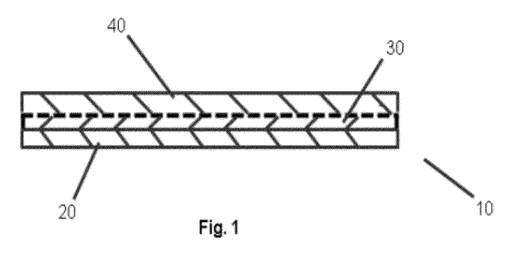

In one embodiment shown in FIG. 1, a medical device 10 has a surface 20 of a biocompatible metal, a primer layer 30 and second biocompatible polymer layer 40. The second biocompatible polymer layer 40 may include a therapeutic agent.

The biocompatible metal is suitably a medical grade alloy that is used in forming implant devices; these include for example, without limitation, medical grade stainless steels (e.g. 316L), titanium, nitinol, colbalt-chromium (L-605 or MP 35N) and platinium-chromium (e.g. an alloy comprising about 37% Fe, 33% Pt, 18% Cr, 9% Ni, 2.6% Mo, 0.05% Mn), and mixtures thereof. In some embodiments of the invention the biocompatible metal comprises iron or another metal that will form an organo-metallic complex with functional groups in the primer polymer.

The medical device can suitably be a metal stent, or metal portions of a stent graft, whose coatings are subjected to high stress because of bending and deformation during delivery and deployment. However the invention may have benefits for coating other devices implanted or inserted into the body such as guide wires, needles, pacemakers defibrillators, filters, or the like.

The inventive primer layer is baked after application to the surface of the medical device. The primer layer is baked at a temperature above the glass transition of the coating material and suitably at a temperature in which a “viscous flow” state is achieved. In some embodiments the baking temperature is above 85 degrees Celsius and below 145 degrees Celsius. In some preferred embodiments, the primer layer is baked at 105-130 degrees Celsius, more preferably 110-120 degrees Celsius to reach the improved adherence to the metallic medical device. For instance with a PLGA primer layer, improved adhesion was not observed when the primer layer was baked at 85 degrees Celsius or less and the same primer layer material begins to degrade at temperatures of about 145 degrees Celsius and above.

The primer layer is suitably baked so that the polymer reaches the “viscous flow” state in a vacuum oven depending on the polymer and desired qualities of the primer layer. The time of baking may be adjusted depending on the type of baking device used. Other baking devices known to a person of ordinary skill are also contemplated. Vacuum is used to maintain a dry inert environment. Pressure in the vacuum oven is suitably between 10-50 millibars. Dry inert gases such as nitrogen, helium or argon may also be used in the baking ovens either as a purge or to allow operation at higher pressure. Convection ovens with dry inert gas environments may provide more efficient heat transfer during baking and hence may allow for reduction in baking time.

The embodiments of the composition for a primer layer can be prepared by conventional methods wherein all components are combined, then blended. More particularly, in accordance to one embodiment, a predetermined amount of a thermoplastic polymer is added to a predetermined amount of a solvent or a combination of solvents. The mixture can be prepared in ambient pressure and under anhydrous atmosphere. Heating and stirring and/or mixing can be employed to effect dissolution of the polymer into the solvent.

The solvent should be compatible with the polymer and should be capable of placing the polymer into solution at the concentration desired in the solution. Useful solvents should also be able to expand the chains of the polymer for maximum interaction with the surface of the device, such as a metallic surface of a stent. Examples of solvent can include, but are not limited to, dimethylsulfoxide (DMSO), chloroform, acetone, water (buffered saline), xylene, acetone, methanol, ethanol, 1-propanol, tetrahydrofuran, 1-butanone, dimethylformamide, dimethylacetamide, cyclohexanone, ethyl acetate, methylethylketone, propylene glycol monomethylether, isopropanol, N-methyl pyrrolidinone, toluene and mixtures thereof.

Biocompatible polymers having acid or ester terminated ends and capable of chelating metal ions can be used for the primer material, and in some embodiments are preferred. Examples of biocompatible primer polymers include poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonates), poly(iminocarbonate), copoly(ether-esters) (e.g. PEO/PLA), polyalkylene oxalates and polyphosphazenes.

In some preferred embodiments the primer polymer is a polylactic acid or poly(lactate-co-glycolide) (PLGA). These polymers may have different molecular weights and lactic acid (LA) to glycolic acid (GA) ratios. In some embodiments a low molecular weight polymer (for instance an Mw of about 2000) may provide better performance, potentially due to more available acid end groups than a higher molecular weight (for instance about 100,000). As to LA/GA ratio, a ratio of from about to 100/0 to about 50/50, are generally suitable with a higher LA to GA ratio generally providing a longer degradation time of the polymer. An LA/GA ratio in the range of 85:15 is preferred.

The primer layer can be applied to the medical device by any known method in the art including, but not limited to spraying, dipping painting, rolling, laser coating, sponge painting and so forth.

The second layer can be applied using the same techniques as the application of the primer layer. The choice of polymer for the second biocompatible layer can be the same as or different from the selected polymer for the primer layer. The use of the same polymer significantly reduces or eliminates interfacial incompatibilities, such as lack of an adhesive tie or bond, which may exist with the employment of two different polymeric layers. However, it may be advantageous to use a second polymer different from the first polymer to create the second layer.

The polymer chosen for the second layer must be a polymer that is biocompatible and minimizes irritation to a lumen wall when the device is implanted. The polymer may be either a biostable or a bioabsorbable polymer. Bioabsorbable polymers that could be used include, for example, poly(hydroxyvalerate), poly(L-lactic acid), polycaprolactone, poly(lactide-co-glycolide), poly(hydroxybutyrate), poly(hydroxybutyrate-co-valerate), polydioxanone, polyorthoesters, polyanhydrides, poly(glycolic acid), poly(D,L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphazenes and biomolecules such as fibrin, fibrinogen, cellulose, starch, collagen and hyaluronic acid. Also, biostable polymers with a relatively low chronic tissue response such as polyurethanes, silicones, and polyesters could be used and other polymers could also be used if they can be dissolved and cured or polymerized on the stent such as polyolefins, polyisobutylene and ethylene-alphaolefin copolymers; acrylic polymers and copolymers, vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones; polyvinyl aromatics, such as polystyrene; polyvinyl esters, such as polyvinyl acetate; copolymers of vinyl monomers with each other and olefins, such as ethylene-methyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; rayon; rayon-triacetate; cellulose, cellulose acetate, cellulose butyrate; cellulose acetate butyrate; cellophane; cellulose nitrate; cellulose propionate; cellulose ethers; and carboxymethyl cellulose

The second layer may also contain a therapeutic agent. The therapeutic agent used in the second layer can include any substance capable of exerting a therapeutic or prophylactic effect for a patient. Such substances may include small molecule substances, peptides, proteins, oligonucleotides, and the like. The therapeutic substance could be designed, for example, to inhibit the activity of vascular smooth muscle cells. It can be directed at inhibiting abnormal or inappropriate migration and/or proliferation of smooth muscle cells to inhibit restenosis.

Examples of therapeutic substances that can be used for fabricating the optional second layer include antiproliferative substances such as actinomycin D, or derivatives and analogs thereof (manufactured by Sigma-Aldrich of Milwaukee, Wis., or COSMEGEN available from Merck). Synonyms of actinomycin D include dactinomycin, actinomycin IV, actinomycin I.sub.1 actinomycin X.sub.1, and actinomycin C.sub.1. The active agent can also fall under the genus of antineoplastic, anti-inflammatory, antiplatelet, anticoagulant, antifibrin, antithrombin, antimitotic, antibiotic, antiallergic and antioxidant substances. Examples of such antineoplastics and/or antimitotics include paclitaxel (e.g. TAXOL® by Bristol-Myers Squibb Co., Stamford, Conn.), docetaxel (e.g. Taxotere®, from Aventis S.A., Frankfurt, Germany) methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors such as ANGIOMAX (Biogen, Inc., Cambridge, Mass.). Examples of such cytostatic or antiproliferative agents include angiopeptin, angiotensin converting enzyme inhibitors such as captopril (e.g. Capoten® and Capozide from Bristol-Myers Squibb Co., Stamford, Conn.), cilazapril or lisinopril (e.g. Prinivil® and Prinzide® from Merck & Co., Inc., Whitehouse Station, N.J.); calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), alpha-interferon, genetically engineered epithelial cells, tacrolimus, dexamethasone, and rapamycin and structural derivatives or functional analogs thereof, such as 40-O-(2-hydroxy)ethyl-rapamycin (known by the trade name of EVEROLIMUS available from Novartis), 40-O-(3-hydroxy)propyl-rapamycin, 40-O-[2-hydroxy)ethoxy]ethyl-rapamycin, 40-O-tetrazole-rapamycin. and nitric oxide. An example of an antiallergic agent is permirolast potassium. Mixtures of drugs, for instance two or more of paclitaxel, rapamycin, everolimus, zotarolimus, biolimus A9, dexamethasone and/or tranilast may be employed.

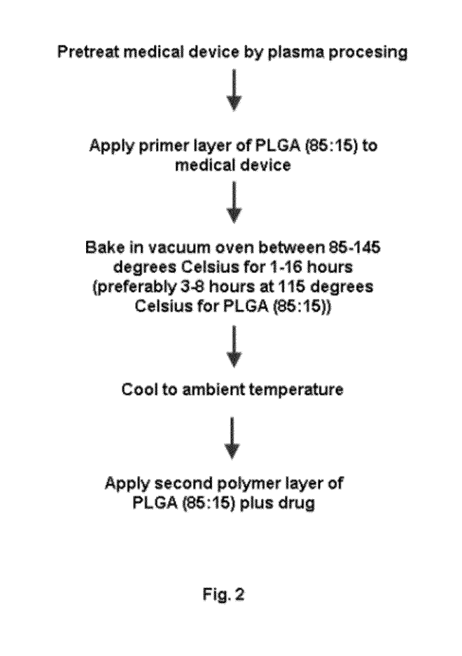

FIG. 2 refers to a method of coating a medical device. The polished surface of a metallic medical device is first prepared by plasma processing using a plasma gas. A primer layer is then applied to the metallic medical device using commonly known techniques. After the desired thickness of the primer layer is achieved, the medical device is then placed in a oven and baked at a suitable temperature for the material as previously described, for instance at 85-140 degrees Celsius so that the primer layer reaches a state of viscous flow. The medical device can be baked for 1-16 hours, depending on the material used as the primer layer. Typically, when the primer layer consists of PLGA (85:15), the medical device is baked for 3-8 hours in a vacuum oven 115 degrees Celsius at 10-50 millibars pressure. After the primer layer is baked, the second biocompatible polymer layer is added to the area previously coated by the primer layer using commonly known techniques, typically from a coating solution. The second layer does not require baking. If desired, a therapeutic agent can be added to the second biocompatible polymer layer.

The invention is illustrated by the following non-limiting examples.

### Example 1

Platinum/chromium alloy (comprising about 37% Fe, 33% Pt, 18% Cr, 9% Ni, 2.6% Mo, 0.05% Mn) stent was subjected to plasma processing during the pre-treatment phase. A primer layer consisting of a polymer coating of PLGA (85:15) was mixed in a solvent consisting of 3:1 DHF:THF to form a polymeric solution. The polymeric solution was roll coated onto the metallic stent until the layer contained between 50-75 μg of polymer. The coating was conducted in ambient temperature and pressure. The metallic stent was then baked in a vacuum oven at 10-50 millibars of pressure at 115 degrees Celsius for 3 hours. The stent was then removed from the oven and allowed to cool to ambient temperature. A second biocompatible layer consisted of PLGA (85:15) in the same solvent as the primer layer. The second layer contained 45% dry weight of drug. The second layer was roll coated onto the stent with a total layer coating of 250 μg/16 mm stent.

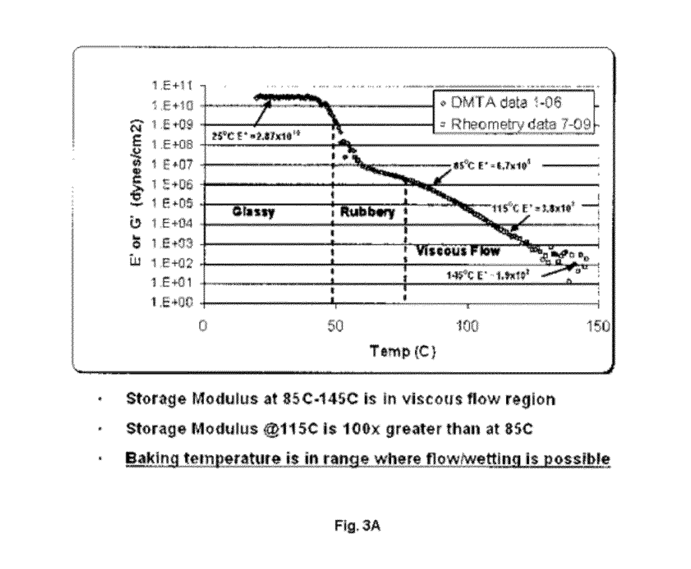

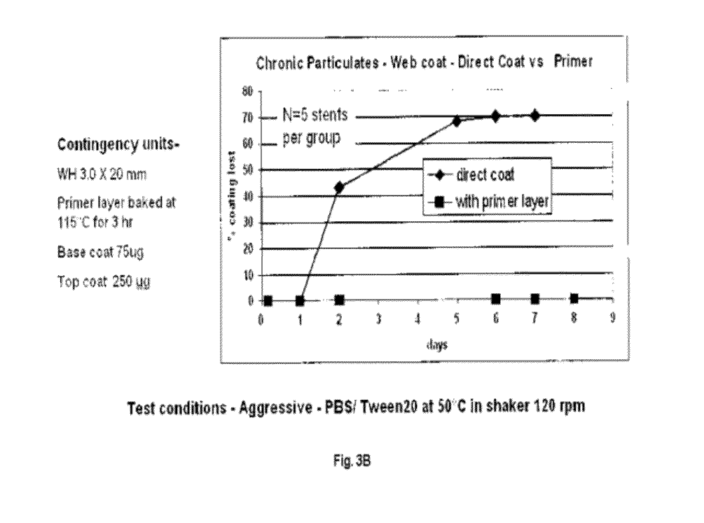

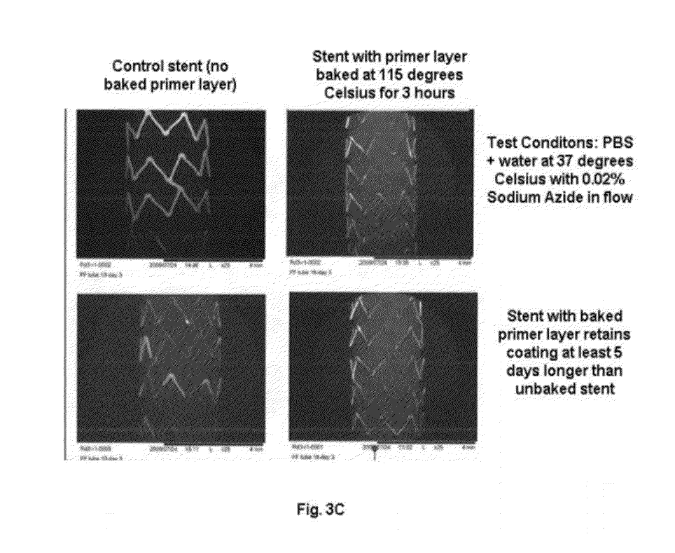

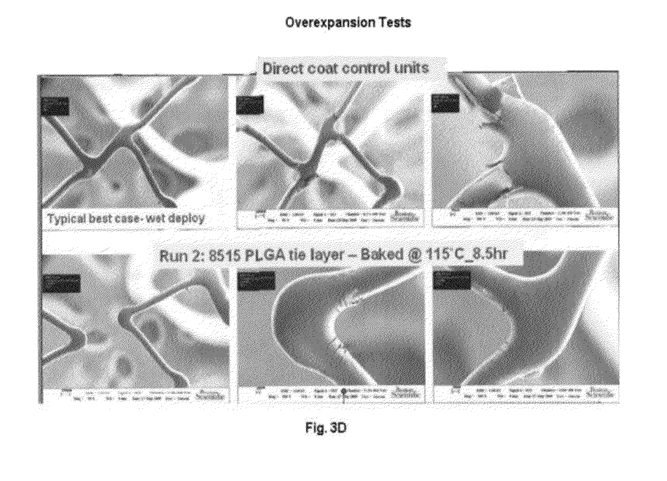

The stents constructed as above were subject to a battery of tests in which they were compared to a stent with a PLGA coating applied directly to the stent surface but not baked. A second layer was then applied to both stents. FIG. 3B shows the results of one such test. FIG. 3B shows the stents with the baked primer layer retained up to 70% more coating after 7 days than the stents lacking the baked primer layer.

FIG. 3C shows the results of stent having a baked primer layer and a stent without a baked primer layer subjected to a flow field test. The stents were baked at 115 degrees Celsius for 3 hours. The same second layer was added to both stents. The stents were subjected to a constant flow of PBS+water at 37 degrees Celsius with 0.02% Sodium Azide. After three days, the unbaked stent was devoid of a coating, while the stents with a baked primer layer retained their coating in a good condition up to 8 days and longer is some instances.

FIG. 3D shows the results of a typical over expansion test. Stents having a baked primer layer were compared to unbaked stents. The same second layer was added to both types of stents. The baked stents contained a PLGA primer layer (85:15) and baked at 115 degrees Celsius for 8.5 hours. When the baked stents were over expanded in both a wet and dry condition, the baked stents showed significantly less cracking in the coating as compared to the unbaked stents.

### Example 2

A 316 L stainless steel stent was coated with a primer in the manner of Example 1 and then the coating encased in a crosslinkable acrylic composition that was thoroughly cured. A portion of the encased coating was peeled from the stent mechanically. The peeled coating removed a portion of the primer layer at its interface with the stent and this portion when subjected to TOF_SIMS (Time of Flight-Secondary Ion Mass Spectroscopy) revealed iron species on the exposed primer interface surface. ICP (inductively coupled Plasma) verified that that the iron came from the stent and not composition used to encase the coating, indicating that an organo-metallic complex is achieved in the baked primer. These results are taken as evidencing a substrate-iron complex by the baked primer layer.

The above disclosure is intended to be illustrative and not exhaustive. This description will suggest many variations and alternatives to one of ordinary skill in this art. All these alternatives and variations are intended to be included within the scope of the claims where the term “comprising” means “including, but not limited to”. Those familiar with the art may recognize other equivalents to the specific embodiments described herein which equivalents are also intended to be encompassed by the claims.

Further, the particular features presented in the dependent claims can be combined with each other in other manners within the scope of the invention such that the invention should be recognized as also specifically directed to other embodiments having any other possible combination of the features of the claims. For instance, for purposes of claim publication, dependent claims 2-17 may be taken as alternately presented as method claims depending from claim 18, and dependent claims 5-17 may alternatively be taken as depending from claim 20.

This completes the description of the preferred and alternate embodiments of the invention. Those skilled in the art may recognize other equivalents to the specific embodiment described herein which equivalents are intended to be encompassed by the claims attached hereto.

What is claimed is:

1. An implantable device comprising a metallic substrate having a coating over at least a portion of the substrate, the coating comprising:
(a) a drug-free baked primer layer comprising a thermoplastic polymer adjacent the substrate, and
(b) a second layer comprising a biocompatible polymer disposed over at least a portion of the area of said implantable device covered by said primer layer.

2. The implantable device according to claim 1 where the implantable device is a stent.

3. The implantable device according to claim 2 wherein the metallic substrate is an alloy comprising iron.

4. The implantable device according to claim 3 wherein the thermoplastic polymer terminates in ends having a functional group capable of forming an organo-metallic complex, where said polymer forms an organo-metallic complex with iron species of said alloy comprising iron.

5. The implantable device according to claim 3 wherein the thermoplastic polymer has acid terminated ends and forms an organo-metallic complex with iron species of said alloy comprising iron.

6. The implantable device according to claim 3 wherein the thermoplastic polymer has ester terminated ends and forms an organo-metallic complex with iron species of said alloy comprising iron.

7. An implantable device according to claim 1 where said primer layer has been baked on said metallic substrate at a temperature that is above 85 degrees Celsius and less than 145 degrees Celsius.

8. An implantable device according to claim 1 where said primer layer has been baked on said metallic substrate at a temperature in the range of 105-130 degrees Celsius.

9. An implantable device according to claim 1 where said primer layer has been baked on said metallic substrate at a temperature in the range of 110-120 degrees Celsius.

10. The implantable device according to claim 1 where the thermoplastic polymer is selected from the group consisting of poly(lactic acid), polycaprolactone, poly(lactide-co-glycolide) or a combination thereof.

11. The implantable device according to claim 1 wherein the thermoplastic polymer is a poly(lactide-co-glycolide) having acid terminal groups.

12. The implantable device according to claim 11 wherein said poly(lactide-co-glycolide) has a LA/GA ratio of from about 50:50 to about 85:15.

13. The implantable device according to claim 11 wherein the poly(lactide-co-glycolide) has a molecular weight (Mw) of no more than about 2000.

14. The implantable device according to claim 1 where the polymer of the second layer is the same polymer as the thermoplastic polymer, but has not been baked.

15. The implantable device according to claim 1 where the second layer contains a therapeutic agent.

16. The implantable device according to claim 15 where the therapeutic agent comprises paclitaxel, rapamycin, everolimus, zotarolimus, biolimus A9, dexamethasone and/or tranilast.

17. The method according to claim 1 where the second layer is devoid of a therapeutic agent.

18. A method of forming a coating on an implantable device comprising:
(a) providing a primer layer comprising a thermoplastic polymer on a metal surface of at least a portion of said implantable device;
(b) baking said implantable device after application of said primer layer at a temperature and time effective to adhere the thermoplastic polymer to the substrate; and
(c) subsequently providing a second layer comprising a biocompatible polymer disposed over at least a portion of the area of the implantable device previously covered by said primer layer.

19. The method according to claim 17 where the baking is performed in an inert environment provided by a vacuum and/or a dry inert gas.

20. An implantable metal stent comprising a metallic substrate comprising iron having a coating over at least a portion of the substrate, the coating comprising a baked primer layer comprising a polymer capable of forming and organo-metallic complex adjacent the substrate, the polymer being disposed on a surface of at least a portion of said implantable device which forms an organo-metallic complex with iron species of the substrate.
